(19) United States
(12) Patent Application Publication
(10) Pub. No.: US 2005/0154441 A1
(43) Pub. Date: Jul. 14, 2005

(54) Introducer

(75) Inventors: Darin G. Schaeffer, Bloomington, IN (US); Michael C. Hoffa, Ellettsville, IN (US)

Correspondence Address: Brinks Hofer Gilson & Lione, P.O. Box 10395, Chicago, IL, 60610, US

(73) Assignee: Cook Incorporated

(21) Appl. No.: 10/756,851

(22) Filed: Jan. 14, 2004

(51) Int. Cl. A61F 2/06

(52) U.S. Cl. 623/1.11

(57) ABSTRACT

A stent introducer is provided for introducing a stented catheter through a hemostatic valve. The stent introducer is inserted into the hemostatic valve to open the valve member and provide an open passageway for the stented catheter. The stent introducer is made from flexible plastic and has a slot that extends along the entire length of the stent introducer. Advantages of the stent introducer include ease of use and improved protection for the stented catheter during introduction.

### BACKGROUND

The present invention relates generally to medical devices and particularly to a device for introducing a stent through a hemostatic valve.

The use of stents to treat various organs, such as the vascular system, colon, biliary tract, urinary tract, esophagus, trachea and the like, has become common in recent years. Currently, stents are useful in treating blockages, occlusions, narrowing ailments and other similar problems that restrict flow through a passageway. One common medical procedure in which stents are used involves implanting an endovascular stent into the vascular system. Stents have been shown to be useful in treating various vessels throughout the vascular system, including both coronary vessels and peripheral vessels (e.g., carotid, brachial, renal, iliac and femoral).

The use of stents in coronary vessels, however, has drawn particular attention from the medical community because of the growing number of people suffering from heart problems associated with stenosis (i.e., narrowing of a vessel). This has lead to an increased demand for medical procedures to treat such problems. The widespread frequency of heart problems may be due to a number of societal changes, including the tendency of people to exercise less while eating more unhealthy foods, in conjunction with the fact that people generally now have longer life spans than previous generations. Stents have become a popular alternative for treating coronary stenosis because stenting procedures are considerably less invasive than previous procedures. Traditionally, stenosis of the coronary arteries has been treated with bypass surgery. In general, bypass surgery involves splitting the chest bone to open the chest cavity and grafting a replacement vessel onto the heart to bypass the blocked, or stenosed, artery. However, coronary bypass surgery is a very invasive procedure that is risky and requires a long recovery time for the patient.

To address the increased need for coronary vessel treatments, the medical community has turned to stenting procedures, in combination with balloon angioplasty, to avoid the problems associated with traditional bypass surgery. Typically, stenting procedures are performed using a balloon-tipped catheter with a ductile stent compressed onto the balloon (also referred to as a stented catheter). Normally, the manufacturer assembles the balloon-tipped catheter and stent together to make a stented catheter. Thus, the physician is provided with a pre-assembled package that is essentially ready to use. The physician performs the stenting procedure by introducing the stented catheter into a peripheral vessel (commonly one of the leg vessels) and threading the catheter to the narrowed part of the coronary vessel that is to be treated. Once the balloon is positioned at the narrowed part of the vessel, the balloon is expanded by pumping a mixture of saline and contrast solution through the catheter to the balloon. This simultaneously dilates the passageway of the vessel and expands the stent against the inner wall of the vessel. The balloon is then deflated, and the balloon-tipped catheter is retracted from the vessel. The stent is thus left permanently implanted in its expanded state at the desired location in the vessel. Therefore, the stent provides a permanent support structure within the vessel that prevents the treated portion of the vessel from collapsing back to its pre-dilated condition.

One area of particular concern with this type of procedure is proper introduction of the stented catheter into the peripheral vessel. Typically, stented catheters are introduced into peripheral vessels with a hollow introduction sheath which has a hemostatic valve at the proximal end thereof. The sheath is inserted by the physician into the peripheral vessel so that the sheath extends inside the vessel and has a portion remaining outside of the patient's body, thereby providing a conduit to the vessel. The hemostatic valve remains outside of the patient's body and prevents pressurized blood in the vessel from draining out through the introduction sheath during the stenting procedure. Thus, once the introduction sheath has been inserted into the peripheral vessel, the introduction sheath essentially provides the physician with an access port to the vascular system.

Many different types of hemostatic valves are used with introduction sheaths. However, in general terms, most hemostatic valves have a soft valve member with an opening formed therethrough. Various types of endovascular medical instruments, including stented catheters, are introduced through the hemostatic valve by pushing the instrument through the valve opening. The soft valve material permits the opening to widen as needed to pass instruments through. At the same time, the valve material squeezes around the instrument to maintain a seal that prevents blood from leaking out. However, there are many problems involved with introducing a stented catheter directly through a hemostatic valve.

One risk associated with direct introduction of stented catheters through a hemostatic valve is that the stent may shift on the catheter during introduction. Specifically, under certain situations, the valve member may apply so much pressure to the stent while the stent is being pushed through the valve opening that the stent may slide on the balloon towards the proximal end of the catheter. If the stent does shift during introduction, at least four different scenarios are possible.

In the first scenario, the stent may be pushed and/or pulled all the way off the balloon during introduction. This is usually an obvious situation to the physician, and the physician will typically respond by withdrawing the balloon catheter and restarting the procedure with a new stented catheter. The result is that the total time for the stenting procedure is increased and extra expense is incurred for a new stented catheter.

In the second scenario, the stent may be pushed and/or pulled so far along the balloon that the proximal end of the stent is either entirely or nearly off of the proximal end of the balloon. This situation can be especially dangerous if the physician does not notice the problem before expanding the balloon at the stenosed portion of the vessel. If the physician expands the balloon in this scenario, the proximal end of the stent will not expand or will expand only minimally. When this occurs, the physician may not be able to retract the balloon catheter through the proximal end of the stent even when the balloon is fully deflated. Once this problem is encountered, there are few solutions, and some physicians may choose to simply pull on the balloon catheter to withdraw the catheter and the partially expanded stent, while hoping to minimize damage to the vessel.

In the third scenario, the stent may shift so that the proximal end of the stent is still on the balloon but positioned significantly off the working length of the balloon. The working length of a stenting balloon generally refers to the center section of the balloon where the balloon inflates to an equal diameter along the entire center section. However, stenting balloons also have non-working portions at the distal and proximal ends of the balloon. The non-working portions inflate unequally between the diameter of the catheter and the diameter of the working length. Therefore, if the proximal end of the stent is shifted onto the non-working portion of the balloon, the balloon will only partially expand the proximal end of the stent at the stenosed area that is to be treated. As a result, the proximal end of the stent will fail to contact the vessel wall, and the opening through the proximal end of the stent will be narrowed. If the physician notices this problem during the stenting procedure, the physician may attempt to remedy this situation by repositioning the balloon after the first inflation so that the working length of the balloon is at the narrowed end of the stent. The physician may then inflate the balloon a second time in order to fully expand the proximal end of the stent. While the problem may be repaired as described, extra time is required and the vessel must be dilated twice with the balloon.

In the fourth scenario, the stent may be shifted so that the proximal end of the stent is positioned only a small distance off of the working length of the balloon. In this case, the proximal end of the stent will similarly fail to fully expand, but the failure may be imperceptible to the physician. If the physician does not notice this condition during the procedure, the stent will remain permanently implanted with a narrowed opening at the proximal end. Over time, this narrowed area of the stent is likely to suffer from stenosis, thereby defeating the purpose of the original procedure.

In addition to the risks described above, other problems are also possible when introducing stented catheters directly through a hemostatic valve.

One possible problem is that the stent may puncture the balloon during introduction due to the pressure exerted on the stent by the valve member. The consequences of this possibility are unpredictable and may vary from minor to severe.

Another problem relates to coated stents, which are becoming very popular in the stent field. Coated stents usually consist of a metallic stent that has been coated with a drug or other material to prevent restenosis or provide some other therapeutic effect. Normally, coatings like these are applied to the outer surface of the stent so that the coating will come into contact with the vessel wall once the stent is expanded. These coatings are typically crucial to an effective treatment of the vessel and are often very expensive. However, direct introduction of coated stents through a hemostatic valve may result in the coating being damaged. Such damage may occur because the valve member rubs against the outer surface of the stent during introduction, which can result in the coating be scraped off. This problem may often go unnoticed by physicians during the stenting procedure. As a result, the patient may fail to receive a full amount of the prescribed drug.

To avoid the above-described risks and problems, some manufacturers provide physicians with a stent introducer along with the stented catheter. In general, a stent introducer is a special instrument used to open up hemostatic valves during introduction of stented catheters. One of the purposes of stent introducers is to minimize contact between the valve member of the hemostatic valve and the stent. Several types of stent introducers are known, but all of the currently known stent introducers have problems associated with them.

Accordingly, it is apparent to the inventor that a stent introducer is desired which is easy to use and protects a stent during introduction through a hemostatic valve. Therefore, a solution is described more fully below that solves these and other problems.

### SUMMARY

A stent introducer is provided which is made from a flexible plastic material. The stent introducer has a slot that extends through the wall of the stent introducer. The slot further extends along the entire length of the stent introducer. One advantage of the stent introducer is that it is easier to use than prior art stent introducers, thereby encouraging physicians to use the stent introducer instead of resorting to direct introduction. Another advantage of the stent introducer is that it provides almost complete protection from the hemostatic valve member. Additional details and advantages are further described below.

### BRIEF DESCRIPTION OF SEVERAL VIEWS OF THE DRAWINGS

The invention may be more fully understood by reading the following description in conjunction with the drawings in which:

FIG. 1 is a perspective view of a stented balloon catheter being introduced through a hemostatic valve without using a stent introducer;

FIG. 2 is a cross-sectional view of the stented balloon catheter being introduced through the hemostatic valve without using a stent introducer;

FIG. 3 is a perspective view of a first prior art stent introducer, the first prior art stent introducer being made of metal and not having a slot;

FIG. 4 is a perspective view of the first prior art stent introducer inserted through the hemostatic valve;

FIG. 5 is a perspective view of a second prior art stent introducer, the second prior art stent introducer being made of metal and having a slot;

FIG. 6 is a perspective view of the second prior art stent introducer inserted through the hemostatic valve;

FIG. 7 is a perspective view of a third prior art stent introducer, the third prior art stent introducer being made of plastic and not having a slot;

FIG. 8 is a perspective view of the third prior art stent introducer inserted through the hemostatic valve;

FIG. 9 is a side elevational view of one embodiment of a stent introducer according to the present invention;

FIG. 10 is a front elevational view of one embodiment of the stent introducer according to the present invention;

FIG. 11 is a cross-sectional view of another embodiment of the stent introducer according to the present invention, showing a sheath in a free state;

FIG. 12 is a cross-sectional view of the embodiment shown in FIG. 11, showing the sheath closed;

FIG. 13 is a cross-sectional view of another embodiment of the stent introducer according to the present invention, showing a sheath in a free state;

FIG. 14 is a perspective view of the stent introducer according to the present invention;

FIG. 15 is a perspective view of the stent introducer according to the present invention, showing the stent introducer installed on a stented balloon catheter in preparation for insertion into the hemostatic valve;

FIG. 16 is a perspective view of the stent introducer according to the present invention, showing the stent introducer inserted through the hemostatic valve;

FIG. 17 is a perspective view of the stent introducer according to the present invention, showing the stent introducer being removed from the catheter; and

FIG. 18 is a close-up perspective view of one embodiment of the stent introducer according to the present invention, showing the stent introducer being removed from the catheter, the slot of the stent introducer being narrower than the catheter in this embodiment.

### DETAILED DESCRIPTION

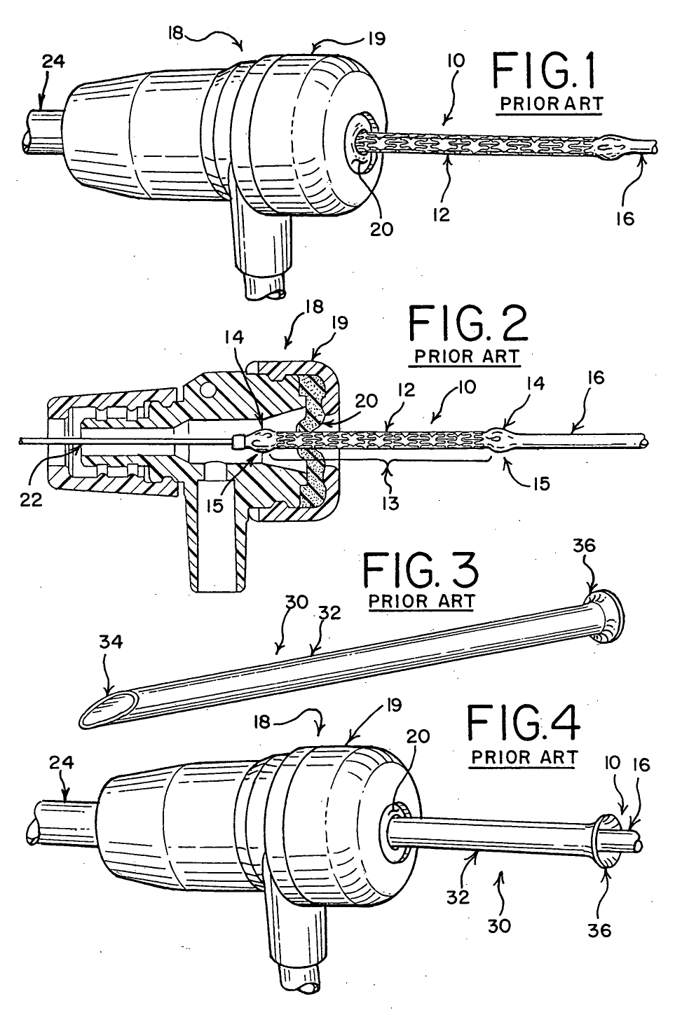

Referring now to the drawings, and particularly to FIGS. 1 and 2, a stented catheter 10 is shown being directly introduced through a hemostatic valve 18. The hemostatic valve 18 serves as the proximal end of an introduction sheath 24. The structure and function of introduction sheaths and hemostatic valves are well known to those in the art. In general, the introduction sheath 24 is hollow and has a hemostatic valve 18 at the proximal end thereof. As those in the art will readily recognize, the introduction sheath 24 serves as an access port to one of the patient's peripheral vessels. Thus, when properly inserted into a peripheral vessel, the distal end of the introduction sheath 24 will extend inside and through the patient's peripheral vessel. The proximal end of the introduction sheath 24 remains outside the patient's body, and the hemostatic valve 18 (also located outside of the patient's body) provides a seal that prevents blood from flowing out of the sheath. The sheath thus provides a conduit to the peripheral vessel through which various endovascular medical instruments may be introduced.

Various types of introduction sheaths 24 are known in the art and may be inserted into the vascular system in numerous ways. One typical procedure for inserting the instruction sheath 24 into a patient's vascular system involves locating the intended vessel with a hollow needle (not shown). Once the vessel is located such that the distal end of the needle is positioned within the vessel, a guidewire 22 is threaded through the needle so that the distal end of the guidewire 22 extends inside the vessel. The needle is then removed. The proximal end of the guidewire 22 is next threaded through a dilator (not shown) which is installed though the introduction sheath 24. When the dilator is installed though the introduction sheath 24, the tip of the dilator typically extends past the distal end of the introduction sheath 24. The distal end of the introduction sheath 24 may or may not be tapered to provide a smooth transition between the outer surface of the dilator and the instruction sheath 24 (only the proximal end of the introduction sheath 24 is shown in the drawings). The dilator and the introduction sheath 24 may then be inserted through the patient's dermis and/or muscular layers and into the intended vessel using the guidewire 22 to direct the dilator and introduction sheath 24. Once inserted, the dilator is withdrawn from the introduction sheath 24. Other endovascular medical instruments may now be introduced through the introduction sheath 24. Although the procedure described above is typical, other procedures for inserting introduction sheaths are also possible.

In order to pass endovascular medical instruments through the introduction sheath 24, an instrument must first be introduced, or inserted, through the hemostatic valve 18. Throughout the drawings, the hemostatic valve 18 that is shown is intended only to be representative of typical hemastatic valves. In fact, there are many different types of hemostatic valves available in the marketplace, and the devices described herein could be used with most hemostatic valves with minimal modifications. In general, a hemostatic valve 18 has a valve housing 19 and a valve member 20. The valve member 20 is typically made from a soft material with elastic properties. Typically, the valve member has an opening (not indicated) that extends through the valve member. One example of the type of valve member that may be used is a disk of silicone with one or more slits extending through the center of the disk. However, there are many other types and variations of valve members that are used in the art.

In FIGS. 1 and 2, the stented catheter 10 is shown partially introduced directly through the hemostatic valve 18. The stented catheter 10 that is shown is typical of those used in the art. In general, a stented catheter 10 is made of flexible materials to allow the catheter 16 to bend and twist through a patient's vascular system. At the distal end of the catheter 16, a deflated balloon 14 is provided with a stent 12 that has been compressed, or crimped, tightly onto the balloon 14 within the working range 13 of the balloon 14. A port (not shown) is provided at the proximal end of the catheter 16 through which saline may be pumped to the balloon 14 in order to inflate the balloon 14 and expand the stent 12 once the catheter 16 has been positioned at the stenosed portion of the vessel that is to be treated. Typically, the manufacturer is especially careful to make sure that no part of the stent 12 is located on the non-working portions 15 of the balloon 14 in order to ensure that the entire length of the stent 12 fully expands when the balloon 14 inflates. Usually, the catheter 16 is hollow so that the catheter 16 may be guided through the vascular system by sliding the catheter 16 along a guidewire 22 that passes through the hollow cavity in the catheter 16. Accordingly, the term “stented catheter” refers generally herein to catheters with a stent mounted thereon which are used to position and implant a stent into a patient's body.

As shown in FIGS. 1 and 2, the valve member 20 of the hemostatic valve 18 directly contacts the stent 12 mounted on the catheter 16. As described above, there are many risks and problems associated with this type of procedure. Despite the risks and problems associated therewith, physicians sometimes still directly introduce stented catheters 10 through hemostatic valves 18 because of the simplicity of this minimalist procedure. Although a few stent introducers are available to reduce the risks and problems associated with direct introduction of stented catheters 10, these alternatives have failed to eliminate the practice of direct introduction. This failure is due in part to the difficulty of using the stent introducers currently available and other problems associated therewith.

Turning now to FIGS. 3 and 4, a first prior art stent introducer 30 is shown. This stent introducer 30 is made of rigid metal, such as stainless steel. The stent introducer consists of a sheath 32 with a continuous circular wall. The distal end 34 of the stent introducer 30 is beveled, and a flange 36 is provided at the proximal end of the stent introducer 30.

FIG. 4 shows the general procedure for using this stent introducer, which is similar to the procedure used for most stent introducers. Typically, the stent introducer 30 is inserted through the valve member 20 of the hemostatic valve 18 prior to introducing the stented catheter 10 through the valve member 20. Once introduced, the outer surface of the stent introducer 30 engages and opens the valve member 20. The inner surface of stent introducer 30 thus provides an open passageway through the valve member 20. The stented catheter 10 may then be passed through the stent introducer 30 and into the introduction sheath 24. Typically, this procedure should be performed reasonably quickly because as soon as the stent introducer 30 opens the valve member 20, blood may flow out through the stent introducer 30. Once the stent 12 has been safely introduced past the valve member 20, the stent introducer 30 is withdrawn from the hemostatic valve 18. The elastic valve member 20 then contracts against the catheter 16 to seal the hemostatic valve 18 to prevent blood leakage.

One of the disadvantages of the first prior art stent introducer 30 is that it cannot be easily installed onto and removed from the stented catheter 10 prior to and after its use. In order to install the stent introducer 30 onto the stented catheter 10, the stent introducer 30 typically has to be threaded onto the distal end of the stented catheter 10 prior to threading the stented catheter 10 onto the guidewire 22. (Threading the stent introducer 30 onto the proximal end of the stented catheter 10 usually will not work because the balloon inflation/deflation valve (not shown) at the proximal end of the stented catheter 10 is too large to pass through the stent introducer 30). After the stent introducer 30 has been used and the distal end of the stented catheter 10 has been introduced, it is normally impossible to completely remove the stent introducer 30 since the stent introducer 30 cannot be threaded off the proximal end of the stented catheter 10 due to the size of the balloon inflation/deflation valve. Thus, the stent introducer 30 is usually left to dangle loosely on the stented catheter 10 during the rest of the stenting procedure.

The difficulty of using the first prior art stent introducer 30 (as well as the other prior art stent introducers) is a serious problem. Faced with a stent introducer that is difficult to use, physicians may be discouraged from using the stent introducer 30, and may choose to avoid these problems entirely by not using the stent introducer 30 at all, resorting instead to the direct introduction procedure. However, as discussed above, the risks and problems associated with direct introduction can be worse. Even when the physician does choose to use the first prior art stent introducer 30, the total time required for the stenting procedure will likely be increased due to the extra effort required to work with the stent introducer 30. The awkwardness of the stent introducer 30 may also distract the physician and cause other problems.

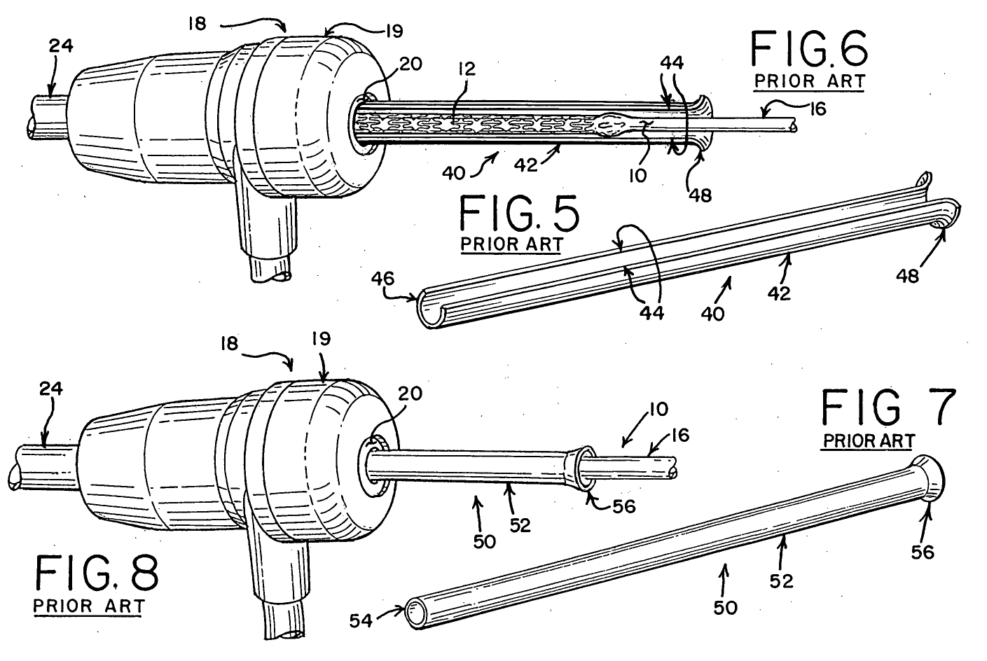

Turning now to FIGS. 5 and 6, a second prior art stent introducer 40 is shown. This stent introducer 40 is made of rigid metal, such as stainless steel. The stent introducer 40 consists of a sheath 42 with a slot 44 that extends through the wall of the stent introducer 40. The distal end 46 of the stent introducer is blunt, and a flange 48 is provided at the proximal end of the stent introducer 40.

Although this stent introducer 40 has a slot 44, which makes the stent introducer 40 easier to install on and remove from the stented catheter 10, the metal construction frustrates this perceived advantage. Because the stent introducer 40 is made of metal, the stent introducer 40 (along with the slot 44 that extends therethrough) is rigid and inflexible. Since the slot 44 must be fixed in shape and size by the manufacturer, the width of the slot 44 generally needs to be wider than the maximum diameter of the stented catheter 10. In addition, to prevent the possibility of interference, the slot 44 usually must be made wider than the minimum width needed in order to ensure nominal clearance between the slot edges and the stented catheter 10. The manufacturer will also usually make the slot 44 even wider to allow for manufacturing variations.

Because of the overly wide slot 44 that is needed for the metal stent introducer 40, the designer of such a stent introducer 40 will typically be faced with a dilemma. If a small diameter sheath 42 is chosen that closely fits around the stent 12, the slot width will occupy a relatively large proportion of the sheath body 42. That is, since the width of the slot 44 is controlled by the size of the stented catheter 10, a close fitting sheath 42 will leave a larger proportion of the stented catheter 10 unprotected. Conversely, if a larger diameter sheath 42 is chosen, the slot width will occupy a smaller proportion of the sheath body 42, and a larger proportion of the stented catheter 10 will be protected. However, a larger diameter sheath 42 will be more difficult to introduce through a standard sized hemostatic valve 18 and may damage the valve 18 during insertion. In addition, the opening that is created by the stent introducer 40 will be larger, thus resulting in more blood leakage during introduction. Insertion of the stent introducer 40 through the hemostatic valve 18 is made even more difficult by the blunt distal end 46. Thus, the second prior art stent introducer 40 will either leave a significant portion of the stent 12 unprotected from the valve member 20, and/or the stent introducer 40 will be difficult to use because of its size.

Turning now to FIGS. 7 and 8, a third prior art stent introducer 50 is shown. This stent introducer 50 is made of plastic. The stent introducer 50 consists of a sheath 52 with a continuous circular wall. The distal end 54 of the stent introducer is blunt, and a small flange 56 is provided at the proximal end of the stent introducer 50.

Like the first stent introducer 30, the third stent introducer 50 cannot be easily installed onto and removed from the stented catheter 10. In addition, the blunt end 54 of the stent introducer 50 makes the stent introducer 50 more difficult to insert through the hemostatic valve 18. Moreover, the small flange 56 at the proximal end is smaller than the valve member 20 of the hemostatic valve 18. Therefore, there is a risk that the stent introducer 50 could be pushed all the way through the hemostatic valve 18. The small flange 56 in conjunction with the blunt end 54 also makes it possible for physicians to confuse the distal and proximal ends (54 and 56), thereby possibly installing the stent introducer 50 onto the stented catheter 10 backwards. The small flange 56 also makes threading the stented catheter 10 through the stent introducer 50 more difficult.

Turning now to the remaining figures, embodiments of the stent introducer 60 according to the present invention are shown. The stent introducer 60 is made of a flexible plastic material. Teflon is the preferred material for the stent introducer 60, but various other types of plastic materials may also be suitable. Teflon is particularly suitable because it is slicker than other materials, such as stainless steel, and is thus easier to insert into a hemostatic valve 18. The stent introducer 60 comprises a sheath 62 with a slot 64 that extends through the wall of the sheath 64. The slot 64 also extends along the entire length of the stent introducer 60. The distal end 66 of the stent introducer 60 is beveled, with the slot 64 having one end located at the heel of the bevel 66. A flange 68 with an outer periphery that is larger than a hemostatic valve opening is also provided at the proximal end of the stent introducer 60. The inner surface of the flange 68 is beveled, thereby providing a funnel guideway for the stented catheter 10.

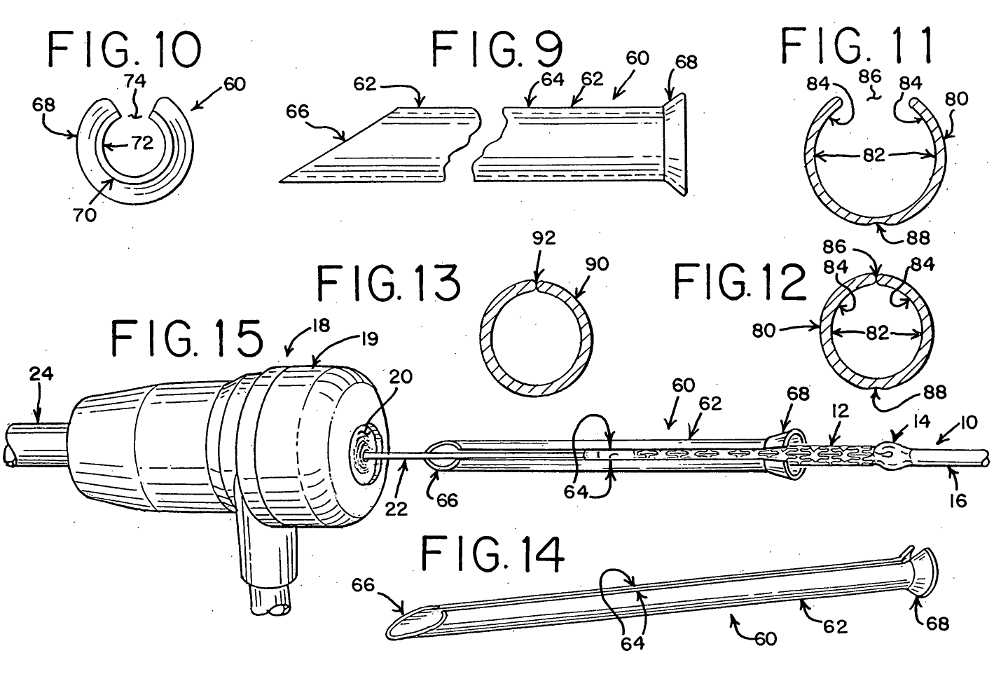

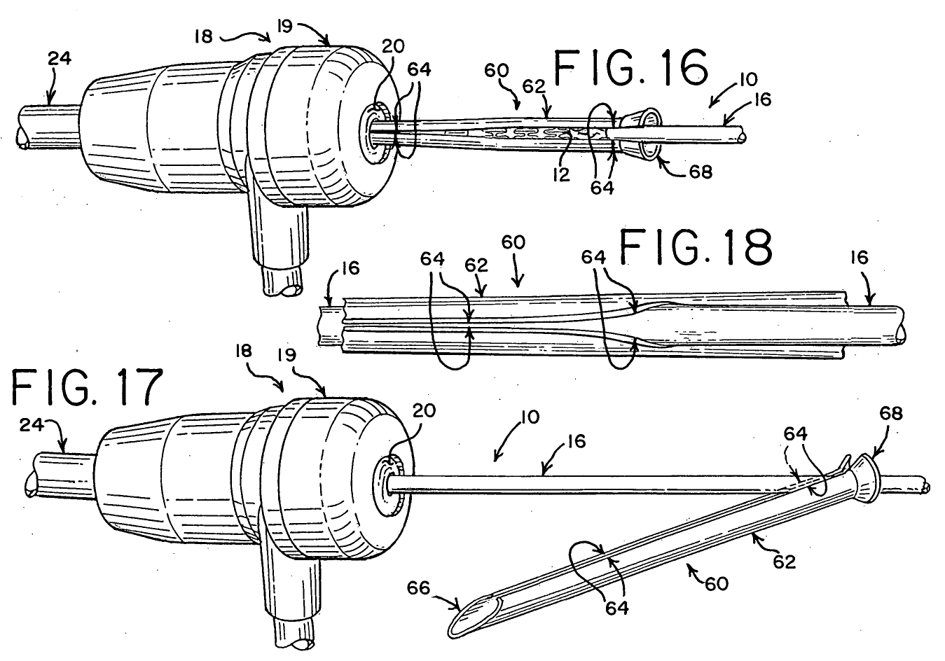

Various constructions of the slot 64 and the sheath 62 are possible depending on the desired characteristics of the stent introducer 60. In one embodiment shown in FIG. 10, the sheath 70 may be designed so that the inner surface 72 of the sheath 70 forms a round cross-section in the free state. In this embodiment, the width of the slot 74 in the free state may be smaller or larger than the diameter of the stented catheter 10. However, preferably, the width of the slot 74 is less than the diameter of the stented catheter 10 (i.e., either the stent 12 itself or the catheter 16), since the sheath 70 will close to an elliptical shape during insertion through the hemostatic valve 18. In one embodiment, the width of the slot 74 may be about 0.050 inches, which is typically slightly wider than the diameter of the catheter 16 but narrower than the stent 12. In another embodiment, the width of the slot 74 may be about 0.015 inches, which is typically less than about fifty percent of the diameter of the catheter 16. In yet another embodiment, the width of the slot 74 may be minimal (i.e., basically closed). In the last two embodiments, the stent introducer 60 may be installed onto the stented catheter 10 by flexing the slot 74 open and snapping the slot 74 around the catheter 16.

In another embodiment shown in FIGS. 11 and 12, the sheath 80 may be designed so that the inner surface 82 of the sheath 80 forms two half-round cross-sections 84 in the free state. The two half-round cross-sections 84 are connected together at one side and define the sides of the slot 86 at the other side. A hinge 88 may also be provided to connect the two half-round cross-sections 84 together. Preferably, the hinge 88 is integral with the sheath 80 and is formed with a thinner cross-section of the same plastic material used to form the wall of the sheath 80. Various sizes and shapes for the hinge 88 are possible. However, one specific embodiment uses a hinge 88 with a thickness that is about 0.003 inches thinner than the wall of the sheath 80, where the sheath wall thickness is about 0.023 inches thick. One advantage of this embodiment is that the sheath 80 closes to a round shape when inserted through the hemostatic valve 18 as shown in FIG. 12. In this embodiment, the width of the slot 86 may also be smaller or larger than the diameter of the stented catheter 10. For example, in one embodiment, the slot 86 may be significantly wider than the diameter of the stent 12, since the sheath 80 will close to a round shape regardless of the width of the slot 86. This alternative may be useful to provide extra clearance between the width of the slot 86 and the stent 12 to avoid contact between the edges of the slot 86 and the stent 12. In other specific embodiments, the width of the slot 86 may be 0.050 inches, 0.015 inches, substantially closed, or variations thereof as described above.

In another embodiment shown in FIG. 13, the sheath 90 includes a slot 92 that is substantially closed in the free state. Although a hinge may be provided with this embodiment, a hinge is not necessarily needed and the stent introducer 60 may be used without a hinge as shown. As described above, this embodiment will typically be used by snapping the slot 92 around the catheter 16.

It is now apparent that the stent introducer 60 according to the present invention is easier to use than prior art stent introducers. Accordingly, to use the stent introducer 60, the physician threads the stented catheter 10 onto the guidewire 22 and positions the distal end of the stented catheter 10 (i.e., the stent 12 compressed onto the balloon 14) adjacent the hemostatic valve 18. This initial procedure is unchanged from the simple procedure that would be used for direct introduction of the stented catheter 10. The stent introducer 60 is then installed onto the stented catheter 10 by sliding the slot 64 over the stented catheter 10. This may be accomplished either by snapping the slot 64 around the catheter 16 if the slot 64 is narrower than the catheter 16 (shown in FIG. 18) or by simply dropping the slot 64 over the catheter 16 and/or stent 12 if the slot 64 is wider than the catheter 16 or stent 12. The stent introducer 60 is then pushed forward to insert the beveled distal end 66 of the stent introducer 60 into the hemostatic valve 18. The stent introducer 60 is typically inserted until the beveled distal end 66 passes entirely through the hemostatic valve 18 and the slot 64 has been collapsed closed. The flange 68 prevents the stent introducer 60 from being pushed all the way through the hemostatic valve 18 while providing a funnel guideway for the stented catheter 10. The stented catheter 10 is then introduced through the stent introducer 60 until the stent 12 and the balloon 14 have passed entirely through the hemostatic valve 18. Alternatively, the stent introducer 60 and the stented catheter 10 may be moved simultaneously through the hemostatic valve 18 as long as the beveled distal end 66 of the stent introducer 60 leads far enough ahead of the stented catheter 10 to avoid contact between the stented catheter 10 and the valve member 20 of the hemostatic valve 18. Once the stent 12 and the balloon 14 have passed through the hemostatic valve 18, the stent introducer 60 is withdrawn from the hemostatic valve 18. The valve member 20 of the hemostatic valve 18 then contracts onto the outer surface of the catheter 16, thereby sealing the catheter 16 to prevent blood leakage. The stent introducer 60 is then removed from the catheter 16 by sliding or pulling the stent introducer 60 off the catheter 16 through the slot 64. The physician may now continue the stenting procedure as normal.

One advantage of the stent introducer 60 is its ease of use compared to prior art stent introducers. Unlike other stent introducers, the stent introducer 60 does not need to be threaded onto the end of the stented catheter 10. In addition, the stent introducer 60 may be removed easily after its use instead of remaining on the stented catheter 10 to be left dangling during the rest of the stenting procedure. This is particularly important because the difficulty of using prior art stent introducers discourages physicians from using them and may result in some physicians choosing to use the direct introduction procedure instead. However, with the described stent introducer 60, physicians are more likely to recognize the benefits of using the stent introducer 60 and will be encouraged to use the stent introducer 60 because of its easy to use design.

Another advantage of the stent introducer 60 is that it provides nearly complete protection from the hemostatic valve member 20 notwithstanding its ease of use. Because the sheath 62 is made from flexible plastic, the sheath 62 closes from the radial pressure of the valve member 20 during insertion through the hemostatic valve 18. Therefore, the portion of the slot 64 that is inserted into the hemostatic valve 18 substantially closes, thereby providing an open passageway through the hemostatic valve 18 that is almost completely protected from the valve member 20. The physician may also apply additional pressure to close the slot 64 with his fingers or some other device. Thus, the stented catheter 10 may be passed through the stent introducer 60 with minimal or no contact with the hemostatic valve 18. The beveled distal end 66 of the stent introducer 60 also helps in collapsing the slot 64 during insertion by directing pressure from the hemostatic valve member 20 towards the heel of the bevel 66 where the slot 64 begins. As a result, the pressure of the hemostatic valve member 20 is focused on the slot 64 to ensure that the slot 64 closes during insertion. The flexible plastic material of the stent introducer 60 is also advantageous because it causes less damage to the hemostatic valve 18 than other materials, such as steel.

Accordingly, it is now apparent that there are many advantages of the invention provided herein. Although the preferred embodiment of the introducer has been described herein as a stent introducer, the concepts here disclosed may also be adapted to the introduction of other endovascular medical instruments as well. In addition to the many advantages that have been described, it is possible that there are still other advantages that are not currently recognized but which may become apparent at a later time.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein.

1. A stent introducer comprising: a sheath made from a flexible plastic material, said sheath comprising an outer surface and an inner surface, said sheath further having a slot extending though a wall thereof and extending along an entire length of said sheath, wherein said sheath is adapted to be inserted through a hemostatic valve, said outer surface thereby engaging and opening said hemostatic valve and said inner surface providing an open passageway through which a stented catheter is passed to minimize contact between a stent thereon and said hemostatic valve.

2. The stent introducer according to claim 1, further comprising a flange disposed at a proximal end, said flange comprising an outer periphery larger than an opening of said hemostatic valve thereby preventing said proximal end from passing through said hemostatic valve, said flange further comprising a beveled inner surface thereby providing a funnel guideway for said stented catheter.

3. The stent introducer according to claim 1, wherein said sheath comprises a beveled distal end, said slot being disposed at a heel of said bevel.

4. The stent introducer according to claim 1, wherein said inner surface of said sheath forms a substantially round cross-section in a free state.

5. The stent introducer according to claim 1, wherein said inner surface of said sheath forms two substantially half-round cross-sections in a free state, said two half-round cross-sections being connected to each other at one side and said two half-round cross-sections defining said slot at another side, said slot being open in said free state.

6. The stent introducer according to claim 1, wherein said sheath comprises a hinge connecting two adjacent portions of said sheath together.

7. The stent introducer according to claim 6, wherein said hinge comprises a section of said sheath formed from a thinner cross-section of said flexible plastic material than said two adjacent portions.

8. The stent introducer according to claim 1, wherein a width of said slot is less than a diameter of said stented catheter.

9. The stent introducer according to claim 1, wherein a width of said slot is less than fifty percent of a diameter of said stented catheter.

10. The stent introducer according to claim 1, wherein said slot is substantially closed in a free state.

11. The stent introducer according to claim 1, wherein said sheath comprises a hinge connecting two adjacent portions of said sheath together and a width of said slot is greater than a diameter of said stented catheter.

12. The stent introducer according to claim 1, further comprising a flange disposed at a proximal end, said flange comprising an outer periphery larger than an opening of said hemostatic valve thereby preventing said proximal end from passing through said hemostatic valve, said flange further comprising a beveled inner surface thereby providing a funnel guideway for said stented catheter; wherein said sheath comprises a beveled distal end, said slot being disposed at a heel of said bevel.

13. The stent introducer according to claim 12, wherein a width of said slot is less than a diameter of said stented catheter.

14. The stent introducer according to claim 1, wherein said sheath comprises a beveled distal end, said slot being disposed at a heel of said bevel; and a width of said slot is less than a diameter of said stented catheter.

15. The stent introducer according to claim 14, wherein said sheath comprises a hinge connecting two adjacent portions of said sheath together.

16. The stent introducer according to claim 1, wherein said inner surface of said sheath forms two substantially half-round cross-sections in a free state, said two half-round cross-sections being connected to each other at one side and said two half-round cross-sections defining said slot at another side, said slot being open in said free state; and said sheath comprises a hinge connecting said two half-round cross-sections together.

17. The stent introducer according to claim 16, further comprising a flange disposed at a proximal end, said flange comprising an outer periphery larger than an opening of said hemostatic valve thereby preventing said proximal end from passing through said hemostatic valve, said flange further comprising a beveled inner surface thereby providing a funnel guideway for said stented catheter; wherein said sheath comprises a beveled distal end, said slot being disposed at a heel of said bevel; and said hinge comprises a section of said sheath formed from a thinner cross-section of said flexible plastic material than said two half-round cross-sections.

18. The stent introducer according to claim 1, wherein said inner surface of said sheath forms a substantially round cross-section in a free state, and wherein said slot is substantially closed in a free state.

19. The stent introducer according to claim 1, wherein said sheath comprises a beveled distal end, said slot being disposed at a heel of said bevel; said sheath comprises a hinge connecting two adjacent portions of said sheath together and a width of said slot is greater than a diameter of said stented catheter; and said hinge comprises a section of said sheath formed from a thinner cross-section of said flexible plastic material than said two adjacent portions.

20. The stent introducer according to claim 1, wherein said sheath comprises a beveled distal end, said slot being disposed at a heel of said bevel; and said sheath comprises a hinge connecting two adjacent portions of said sheath together.

21. A stent introducer comprising: a beveled distal end, a flanged proximal end, a sheath extending between said beveled distal end and said flanged proximal end, said sheath being made from a flexible plastic material and comprising an outer surface and an inner surface, and a slot extending though a wall and along an entire length thereof, wherein said sheath is adapted to be inserted into a hemostatic valve, said outer surface thereby engaging and opening said hemostatic valve and said inner surface thereby providing an open passageway through which a stented catheter is passed to minimize contact between a stent thereon and said hemostatic valve.

22. A introducer comprising: a sheath made from a flexible plastic material, said sheath comprising an outer surface and an inner surface, said sheath further having a slot extending though a wall thereof and extending along an entire length of said sheath, wherein said sheath is adapted to be inserted through a hemostatic valve, said outer surface thereby engaging and opening said hemostatic valve and said inner surface providing an open passageway through which a endovascular medical instrument is passed to minimize contact between said endovascular medical instrument and said hemostatic valve.

23. The introducer according to claim 22, further comprising a flange disposed at a proximal end, said flange comprising an outer periphery larger than an opening of said hemostatic valve thereby preventing said proximal end from passing through said hemostatic valve, said flange further comprising a beveled inner surface thereby providing a funnel guideway for said endovascular medical instrument.

24. The introducer according to claim 22, wherein said sheath comprises a beveled distal end, said slot being disposed at a heel of said bevel.

25. The introducer according to claim 22, wherein said inner surface of said sheath forms a substantially round cross-section in a free state.

26. The introducer according to claim 22, wherein said inner surface of said sheath forms two substantially half-round cross-sections in a free state, said two half-round cross-sections being connected to each other at one side and said two half-round cross-sections defining said slot at another side, said slot being open in said free state.

27. The introducer according to claim 22, wherein said sheath comprises a hinge connecting two adjacent portions of said sheath together.

28. The introducer according to claim 27, wherein said hinge comprises a section of said sheath formed from a thinner cross-section of said flexible plastic material than said two adjacent portions.

29. The introducer according to claim 22, wherein a width of said slot is less than a diameter of said endovascular medical instrument.

30. The introducer according to claim 22, wherein a width of said slot is less than fifty percent of a diameter of said endovascular medical instrument.

31. The introducer according to claim 22, wherein said slot is substantially closed in a free state.

32. The introducer according to claim 22, wherein said sheath comprises a hinge connecting two adjacent portions of said sheath together and a width of said slot is greater than a diameter of said endovascular medical instrument.

33. The introducer according to claim 22, further comprising a flange disposed at a proximal end, said flange comprising an outer periphery larger than an opening of said hemostatic valve thereby preventing said proximal end from passing through said hemostatic valve, said flange further comprising a beveled inner surface thereby providing a funnel guideway for said endovascular medical instrument; wherein said sheath comprises a beveled distal end, said slot being disposed at a heel of said bevel.

34. The introducer according to claim 33, wherein a width of said slot is less than a diameter of said endovascular medical instrument.

35. The introducer according to claim 22, wherein said sheath comprises a beveled distal end, said slot being disposed at a heel of said bevel; and a width of said slot is less than a diameter of said endovascular medical instrument.

36. The introducer according to claim 35, wherein said sheath comprises a hinge connecting two adjacent portions of said sheath together.

37. The introducer according to claim 22, wherein said inner surface of said sheath forms two substantially half-round cross-sections in a free state, said two half-round cross-sections being connected to each other at one side and said two half-round cross-sections defining said slot at another side, said slot being open in said free state; and said sheath comprises a hinge connecting said two half-round cross-sections together.

38. The introducer according to claim 37, further comprising a flange disposed at a proximal end, said flange comprising an outer periphery larger than an opening of said hemostatic valve thereby preventing said proximal end from passing through said hemostatic valve, said flange further comprising a beveled inner surface thereby providing a funnel guideway for said endovascular medical instrument; wherein said sheath comprises a beveled distal end, said slot being disposed at a heel of said bevel; and said hinge comprises a section of said sheath formed from a thinner cross-section of said flexible plastic material than said two half-round cross-sections.

39. The introducer according to claim 22, wherein said inner surface of said sheath forms a substantially round cross-section in a free state, and wherein said slot is substantially closed in a free state.

40. The introducer according to claim 22, wherein said sheath comprises a beveled distal end, said slot being disposed at a heel of said bevel; said sheath comprises a hinge connecting two adjacent portions of said sheath together and a width of said slot is greater than a diameter of said endovascular medical instrument; and said hinge comprises a section of said sheath formed from a thinner cross-section of said flexible plastic material than said two adjacent portions.

41. The introducer according to claim 22, wherein said sheath comprises a beveled distal end, said slot being disposed at a heel of said bevel; and said sheath comprises a hinge connecting two adjacent portions of said sheath together.
